(19) United States
(12) Patent Application Publication
(10) Pub. No.: US 2008/0275540 A1
(43) Pub. Date: Nov. 6, 2008

(54) Artificial Heart Valve Stent and Weaving Method Thereof

(76) Inventor: Ning Wen, Shanghai (CN)

Correspondence Address: GLOBAL IP SERVICES, 7285 W. Eagle Court, Winton, CA, 95388, US

(21) Appl. No.: 12/093,216

(22) PCT Filed: Nov. 7, 2006
(86) PCT No.: PCT/CN2006/002974

§ 371 Date: May 9, 2008

(30) Foreign Application Priority Data
Nov. 9, 2005 (CN) 200510110144.3
Dec. 23, 2005 (CN) 200510111908.0
Dec. 23, 2005 (CN) 200510111909.5

(51) Int. Cl. A61F 2/06; A61F 2/24

(52) U.S. Cl. 623/1.26; 623/1.2; 623/2.12; 623/2.38

(57) ABSTRACT

An artificial heart valve and weaving method thereof are disclosed. The valve stent includes a tubular stent (10), valve leaflets (33), sealing membranes (351, 354), x-ray opaque markers (311, 312) and flexible connecting loops (41). The middle segment (15) of the net stent (10) is tubular or drum-shaped, or provided with radial protrusion structures (153), or provided with outer annular structures (155), or provided with outer free tongues (156), or provided with radial protrusion structures (153) and outer free tongues (156). The stent can be made by up and down interweaving the same one elastic metal wire, and also can be made by up and down interweaving different elastic metal wires. Moreover, the structure, shape and function of the valve stent are optimized; in radical compression, the valve can be transported to the right place with the help of interventional device; after expansion, fitted with figure of the vascular wall in the radical and axial direction, the artificial heart valve stent will not produce paravalvular leak; even more after implanting, the valve has a normal effect on prevent the slippage of artificial valve, which is caused by the blood reflux through the closed valve.

### FIELD OF THE INVENTION

The present invention relates to a human tissue substitute, especially to artificial heart valve stent and weaving method thereof.

### BACKGROUND OF THE INVENTION

Heart, the most important human organ, is made up left and right parts while each part consists of atria and ventricles. Left and right atria are separated by atrial septum while left and right ventricles are separated by ventricular septum. Four cardiac valves, consisting of tricuspid valve, pulmonary valve, mitral valve and aortic valve, play a crucial role in human blood circulation. The hypoxic blood in the systemic circulation enters the right atrium through vein and the right ventricle through the tricuspid valve in turn. And then the blood is pumped into pulmonary circulation through the pulmonary valve by the right ventricular systole. After the oxygen saturation in the pulmonary circulation, the blood goes back to the left atrium through vein and reaches the left ventricle through mitral valve. In the end, the blood is pumped into the aorta through aortic valve by left ventricular systole and returns to the systemic circulation again. Left and right coronary artery openings are located below the aortic valve. The structures of the four cardiac valves ensure the valves open when blood circulation is in right direction, which reduces heart burden caused by blood backstream, otherwise they will close. However, such structures might lead to some acquired injury or pathological changes of the cardiac valves, for various reasons such as rheumatism, atherosclerosis and so on. In addition, there are some congenital heart diseases such as the tetralogy of Fallot whose remote post-operative effect can also generate the pathological changes of the pulmonary valve. The valvular lesion can cause the valves' functions lose gradually. For example, the valvular insufficiency can lead to blood back-stream, the narrow valves can bring about difficult blood circulation, or both of the two effects. The process mentioned above will make the heart burden so heavily that it will bring about the exhaustion of heart functions. The traditional treatment to the acquired injury or pathological changes of the cardiac valves is to operate a thoracotomy, which is to open the heart to operate the plastics of the valve lesion or artificial cardiac valve replacement with the support of extracorporeal circulation after the heart ceases beating. Current artificial cardiac valve can be classified as two categories: metal mechanical valve and biologic valve. Biologic valve is from processing animal materials such as bovine pericardium, valved bovine jugular vein and porcine aortic valve. The above-mentioned open-heart surgery is characterized as long operation time, high cost, profound wound and high risk. Furthermore, for one thing, the patients need to take a long time to operate anticoagulation therapy after they perform artificial cardiac valve replacement. For another, because of the limited lifespan of the biologic valve materials, patients often need an extra operation.

In order to solve the defects caused by the thoracotomy, people employ the method of percutaneous intervention to implant artificial cardiac valve instead of attempting to operate an open-heart surgery. Currently, there are two kinds of technologies for the interventional artificial cardiac valve.

1. Balloon Expanding Artificial Cardiac Valve

This kind of balloon expanding artificial cardiac valve is a biologic valve. In order to reach the valve's functional mode, we can adopt such an interventional way that is to set the biologic valve on a plastometric stent respectively and compress the valve on a balloon in a radial direction to minify its diameter, implant percutaneously and press the balloon to expand and set the stent.

In 1989, Henning Rud Andersen et. al (Patent No. WO9117720 had first completed the artificial heart valve replacement of porcine aortic valve via duct. (Reference to European Heart Journal 1992 13, 704-708)

In 2000, Philippe Bonhoeffer (Patent No. EP1057460) and Alain Cribier (Patent No. EP0967939) first developed artificial heart valve replacement of pulmonary valve and aortic valve via ducted intervention, respectively.

The disadvantages and problems of balloon expanding artificial cardiac valve: diameter of artificial cardiac valve was determined by the diameter of balloon. If the diameter had not been selected well at the beginning, or after some physiological changes, such as natural growth, pathological vascular ectasias et. al., caliber of natural valve might increase, but if the caliber of artificial valve could not be suitable to increase of the stent's diameter, and artificial valve might be at the risk of loose or slippage. Therefore, the balloon must be reexpanded.

1. Self-Expanding Artificial Cardiac Valve

This kind of artificial valve owns an elastic stent which can expand by itself under radial compression.

Marc Bessler (U.S. Pat. No. 5,855,601) and Jacques Seguin (Patent No. FR2826863, FR2828091) also designed artificial heart valve replacement via duct, but the different with the above method was that they used an elastic deformable stent, which could be self-expanding after radial compression.

The artificial heart valve of Philippe Bonhoeffer (Patent No. EP1281375, US2003036791) utilized an elastic deformable stent, which had contacts at the upper or distal tips, and press at in the both internal sheath and external sheath.

Drum-type stent in the valve's intermediate section, self-expanding and strengthened man-made stent and conjoined implantation device are mentioned in the invention whose Chinese application number for patent of invention is 200410054347.0.

The disadvantages and problems the balloon expanding and self-expanding artificial cardiac valve mentioned above own commonly are as follows:

1. Even with the help of x-ray inspection, interventional self-expanding stent and its implantation device can not be located in the valve's axial upward and backward position easily because the anatomic site can not be judged accurately and the artificial valve become unsteady due to the surging of the blood stream. If the interventional artificial aortic valve locates upward, it will exercise an influence on mitral valve; if it locates backward, it will block the coronary artery opening.

2. The location of the rotation direction of the interventional aortic valve self-expanding stent and its implantation device is not resolved. If the interventional aortic valve rotates in a wrong direction, it will block the coronary artery opening.

3. If patient already has coronary artery bypass, the implanted artificial valve stent will not influence haemoperfusion of bypass opening at aorta ascendens.

4. If self-expanding aortic valve stent of Philippe Bonhoeffer and Jacques Seguin can be successfully implanted, although it can not immediately influence the haemoperfusion of coronary artery after operation, the intermediate segment of stent does not stick to the vascular wall of aortic root, and let blood pass through the meshes of stent, thrombus will form on the one hand, while on the other hand, self-expanding aortic valve stent may change or hinder the interventional treatment and diagnosis of coronary artery in the future.

5. There are some problems below about fixation of valve stent after release of expansion.

a). The impact of systolic and diastolic blood flow will make artificial valve stent move, which is not fixed well

b) Some patients with aortic valve insufficiency, need great valve stent fitted with this problem, because aortic root was pathological expansion before operation.

c) Some patients implanted artificial valve stent had local anatomic changes, such as expansion, which could make valve stent without suitable corresponding changes lose the effective fixation

6 In many cases, after expanding fixation, there are paravalvular leaks of artificial valve stent, which is from valve stent and vascular wall.

7. If switch of valve leaflet contacts metal stent, it will cause the valval abrasion.

8. In order to fix well, the valve stent with great diameter will be adopted. Therefore, valve commissure will bear large stress, leading to the abrasion of valve commissure.

### SUMMARY OF THE INVENTION

The purpose of this invention is to overcome existing technical problems above, provides a new-style artificial heart valve stent, which can not be used in the interventional treatment but also minimally invasive surgery.

The technical scheme of the present invention is a type of artificial heart valve stent, which comprise a tubule-shaped stent with radial deformational ability under expansion and compression. The stent includes upper segment, intermediate segment and lower segment. There are many deformable units formed or wrapped among different netlines of stent. Many arched inflextions can be generated in the tips of stent, which are designed with sealed line-eye separated from deformable units. Switch connected with inside of intermediate segment let blood pass through valve leaflet unidirectionally. Combined line of valve leaflet forms at joint between valve leaflet and stent, while two nearby combined lines of valve leaflet crosslink to generate the valve leaflet commissure. Both inside and outside of stent is covered with sealing membrane, which is extended to intermediate segment of the stent. Moreover, there are many x-ray opaque markers and flexible connecting loops in the stent.

The above artificial heart valve stent, wherein the stent can be made by up and down interweaving the same one elastic metal wire, and also can be made by up and down interweaving different elastic metal wires.

The above artificial heart valve stent, wherein said intermediate segment of stent generate one outside radial protrusion structure, with a great stent opening in the centre. The lunate upper and lower periphery are formed at the joint of radial protrusion structure and stent. Moreover, the lunate upper periphery comprises combined line of valve binding to valve leaflet. The said valve leaflet is consistent with radial protrusion structure and connects with lunate upper periphery of protrudent structure.

The above artificial heart valve stent, wherein said radial protrusion structure in the intermediate segment of stent is one.

The above artificial heart valve stent, wherein said radial protrusion structure in the intermediate segment of stent are two, which were distributed by rotary angle from 90°-180°.

The above artificial heart valve stent, wherein said radial protrusion structure in the intermediate segment of stent are three, which are averagely distributed by circumference along the net stent.

The above artificial heart valve stent, wherein the upper segment of stent shows funnel-shaped.

The above artificial heart valve stent, wherein the periphery of funnel-shaped upper segment is designed with wave-shaped edge, corresponding to the radial protrusion structure in intermediate segment.

The above artificial heart valve stent, wherein the stent comprises the inner layer of stent body with tubule-shaped or with tubular-shaped radial protrusion structure, where the stent is connected with at least one outer tongue structure wrapped by net line. The outer tongue structure and inner layer of stent generate stationary edge at the upper segment, or the joint of the intermediate and upper of stent, which extends from stationary edge to form free edge. Moreover, the free edge overlaps with outer radial protrusion structure on the two parallel surfaces, or at least with the lunate upper periphery.

The above artificial heart valve stent, wherein the number of outer tongue structure is three, which are averagely distributed by circumference along the inner stent.

The above artificial heart valve stent, wherein outer tongue structure, corresponding to inner radial protrusion structure at radial and axial directions, is at the same rotational angle.

The above artificial heart valve stent, wherein said the intermediate segment of stent is inner and outer double-layer tubule-shaped framework, with an outer ringy structure in the inner stent. The outer ringy structure and inner layer of stent generate stationary edge at the upper segment, or the joint of the intermediate and upper of stent, while outer ringy structure ends at the joint of the intermediate segment and upper of stent to form a free edge.

The above artificial heart valve stent, wherein said the stent shows the same size like tubule-shaped, with the opening in the intermediate segment of stent.

The above artificial heart valve stent, wherein the middle segment of stent displays protrudent drum-shaped, with the opening of the middle segment.

The above artificial heart valve stent, wherein there are at least one reinforced fiber in the valve leaflet, originated from the two different commissures or combined lines, and the reinforced fiber connect with the network stent. Moreover, there is at least one reinforced fiber in the sealing membrane, distributed by circumference and connected with network stent.

The above artificial heart valve stent, wherein said sealing loop equipped at the outside in the juncture of upper and middle segment of stent, is flexible half-open tubular net-shape. There are many dot-shaped openings designed opposite to outer and inner surface of valve stent, or trough openings designed opposite to inner surface.

One of the methods, weaved the stent, is to prepare the internal mole fitted with the expanding stent. Used the elastic metal lines as weaving lines, the main weaving points are as follows:

A. It is not to complete the entire stent body until all deformable units are prepared by knitting along outer outlines of internal mole via spiral winder.

B. The different line-segments of weaving lines form the upper and lower cross points, while the location of the same line-segments at the nearby cross commissure, are converse.

C. The weaving lines generate the quadrangle, which are wrapped by the different line-segments into alterable units. And weaving lines turned at the two tips to form arched line-inflextions.

D. According to the need, the sealed line eyes are prepared by at least 360° cyclovergence at the tips or other parts of stent body.

E. With three radial protrusion structure of stent body in the weaving area, the number of deformable units located in the same radial surface of the stent is the multi-times of three.

F. According to the need, x-ray opaque markers are set in the different segments of weaving lines.

The above weaving method of stent, wherein said the seal line eyes are weaved in the same outline surface, or weaved to be vertical with stent body or be in any angle.

The above weaving method of stent, wherein re-weaving at the local or all parts of stent body weaved completely, is to form two-layer or multilayer stents.

The above weaving method of stent, wherein the said weaving lines are single elastic metal line.

The above weaving method of stent, wherein the weaving lines are dual or multiple comprised by many elastic metal lines, including of a single line made by x-ray opaque materials.

The above weaving method of stent, wherein the weaving lines contain many single lines, each of which can be weaved for a stent, while many stents overlay together to form a combined stent.

The above weaving method of stent, wherein outer tongue structure can also be prepared in the stent body weaved in the step A, and the main points of the weaving method are as follows:

a. First, weaving line is weaved from the upper of the complete stent body repetitively. As the angle between stent body and weaving line is equal to 60°, weaving line is separated from stent body and extends a tongue structure, then enter the stent body to knit through the by turning the symmetric opposite direction repetitively. When weaving is nearly third girth of the stent, the above steps are repeated to prepare three outer tongue structures. Finally a segment of weaving line enters into stent body and reknit repetitively near to the lower end of stent.

b. The weaving out and in points extended from the stent body are dominated in the same radial surface. The distance between out point and in point is nearly third girth of circle, and free edge of outer tongue structure is dominated in the juncture of the upper segment and middle segment of stent body.

The above weaving method of stent, wherein said main point a, extended from the stent body, weaving line can at least generate a 360° loop and a further semi loop, which have the same radian. Moreover, the part of loop comprises tongue structure with semi loop.

The above weaving method of stent, wherein loop separated from the stent body is in full free state. Or the lower segment of loop is weaved into the stent body

The above weaving method of stent, wherein said main point a, when a tongue structures is wrapped by weaving lines, sealed line eyes generate by wrapping at least 360° circle in arched top, and the dual line segments of sealed line eyes are set by the mark loop of impervious x-ray.

The above weaving method of stent, wherein tongue structures and stent body are weaved the same braided line.

The above weaving method of stent, wherein the tongue structures and stent body weaved the different braided line.

### BRIEF DESCRIPTION OF THE DRAWINGS

As the following detailed description of cases about artificial heart valve stent of present invention is considered in conjunction with the following drawings, a better understanding of the present invention can be obtained, including of purpose, virtue and specific characteristic. wherein the figures are as follows:

FIG. 1, wherein the artificial heart valve stent of present invention, is a three-dimensional perspective of tubule-shaped valve stent.

FIG. 1a of the artificial heart valve stent in present invention is a planar graph of the valve with single layer structure

FIG. 2 of the artificial heart valve stent in present invention is a three-dimensional perspective of the drum-shaped valve stent in the middle segment.

FIG. 3 of the artificial heart valve stent in present invention is a three-dimensional perspective of the valve with radial protrusion structure in the middle segment.

FIG. 3a is an elevation of the valve shown in the FIG. 3.

FIG. 3b is top elevation of the FIG. 3a.

FIG. 3c is bottom elevation of the FIG. 3a.

FIG. 3d is side elevation of the FIG. 3a

FIGS. 3e and 3f are a schematic diagram in up and down cross section along side axis bx of FIG. 3b.

FIG. 4 of the artificial heart valve stent in present invention is a three-dimensional perspective of the valve with tubule-shaped dual-layer structure in the segment.

FIG. 4a is a planar graph of the dual-layer weaving structure in the valve of FIG. 4

FIG. 5 of the artificial heart valve stent in present invention is a three-dimensional perspective of the valve with free tongue in the segment.

FIG. 5a is a planar graph of the dual-layer weaving structure in the valve of FIG. 5

FIG. 5b is bottom elevation of the valve stent of the FIG. 5

FIG. 6 of the artificial heart valve stent in present invention is a three-dimensional perspective of the valve with radial protrusion structure and free tongue in the middle segment.

### DETAIL DESCRIPTION OF THE INVENTION

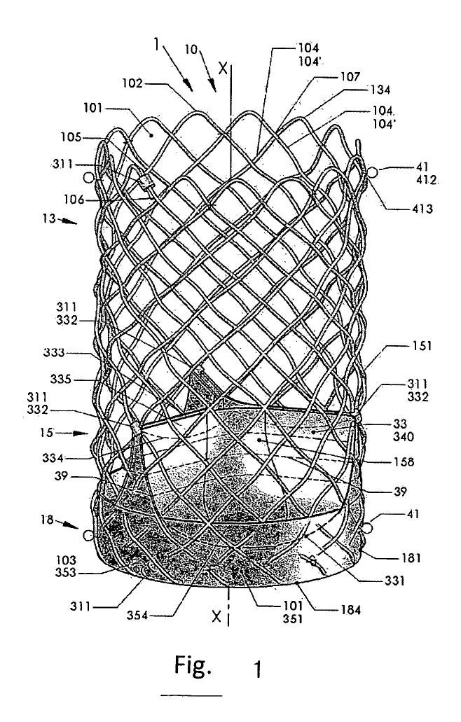

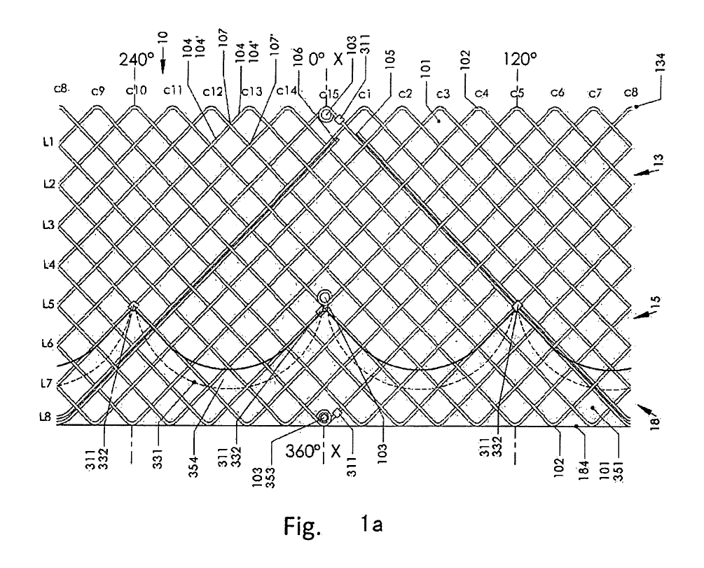

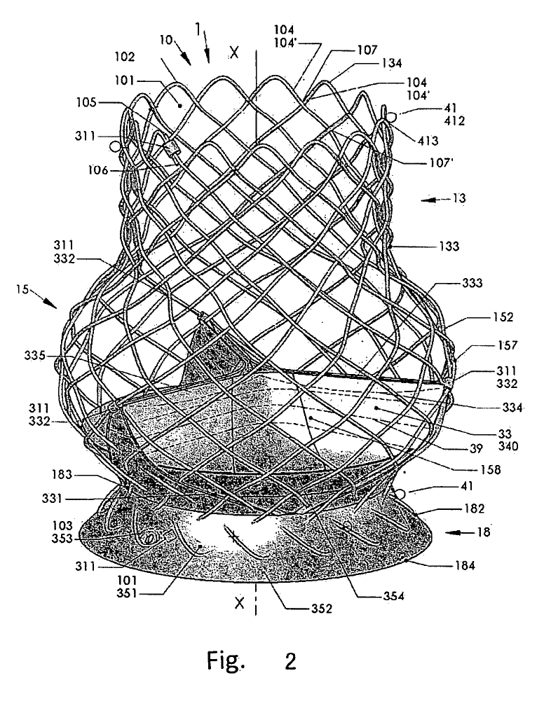

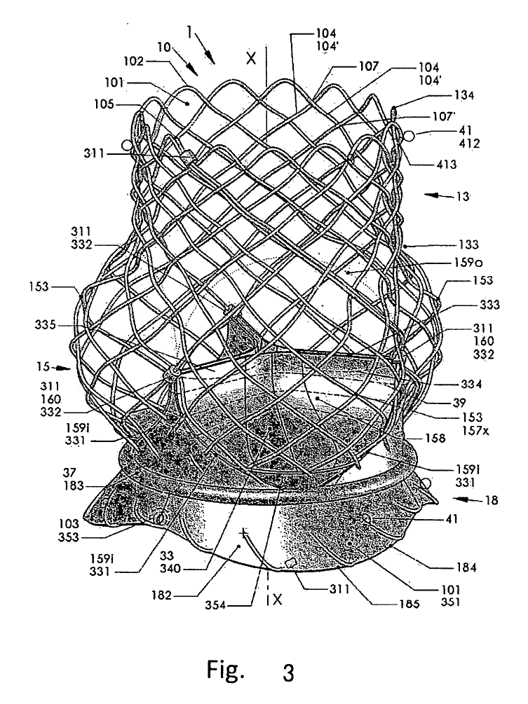

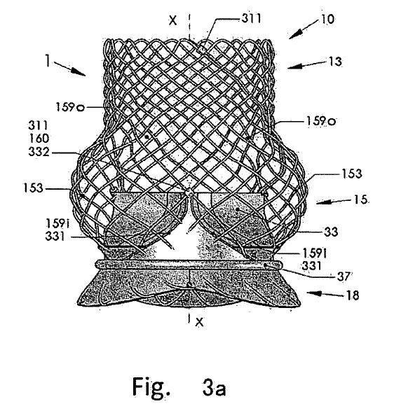

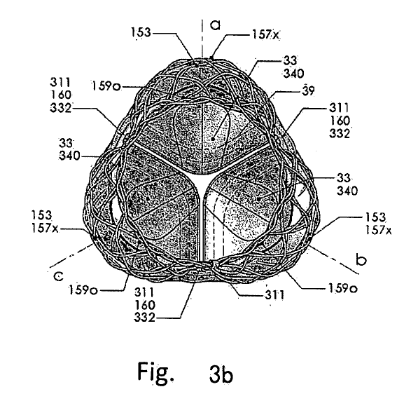

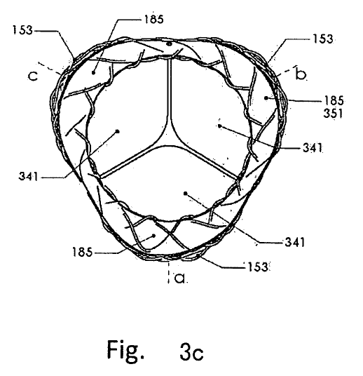

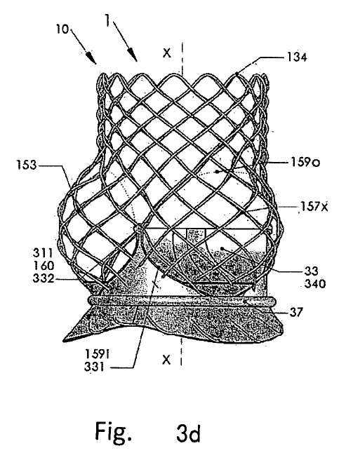

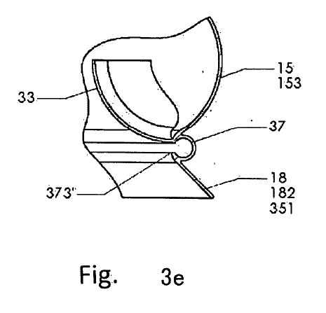

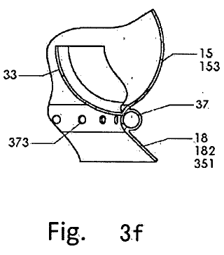

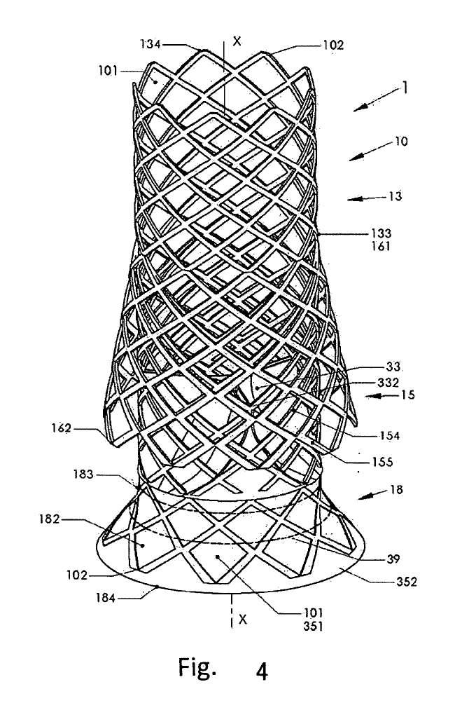

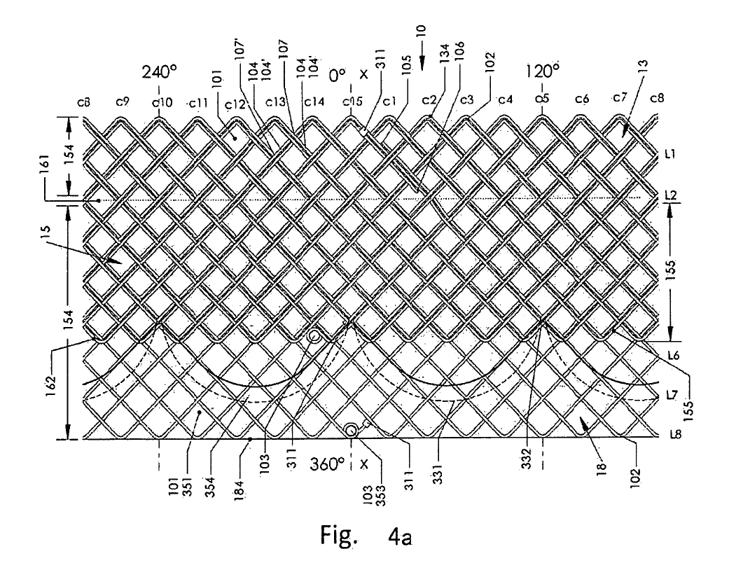

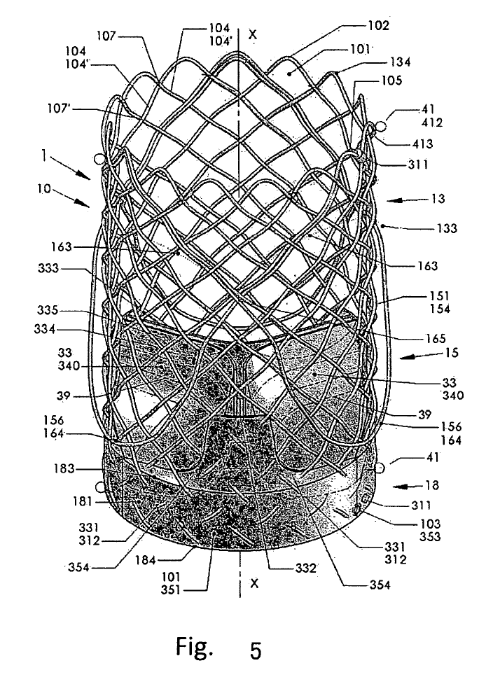

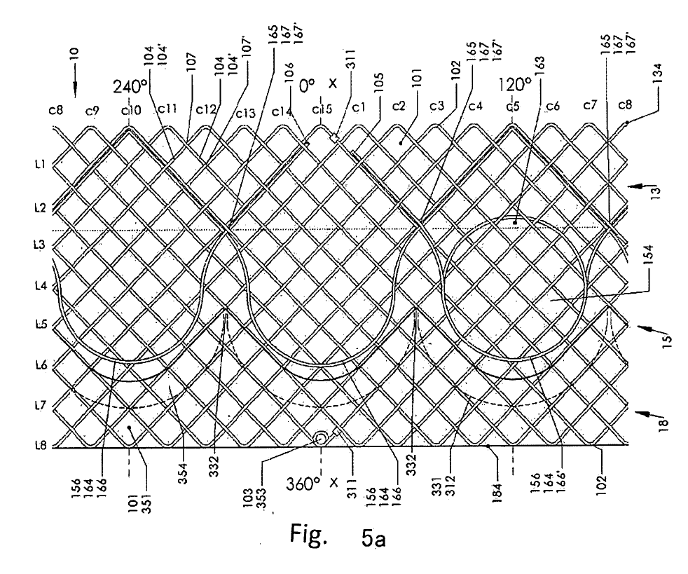

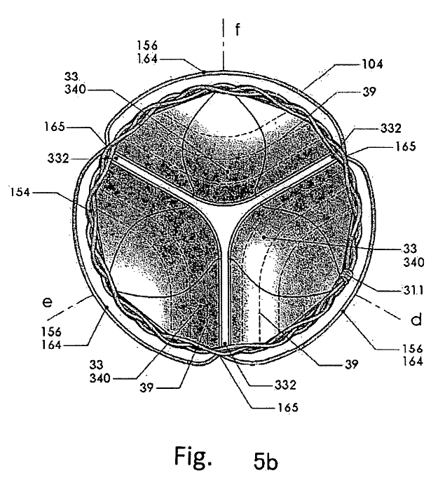

Referring to FIG. 1, combining with FIG. 2, FIG. 3, FIG. 4 and FIG. 5, an artificial heart valve stent in present invention contains: radial deformable self-expanding network stent (10), the x-ray opaque markers (311,312), the valve leaflet (33), sealing loop (37), synthetic intramembrane reinforced fiber (39) and flexible connecting loops (41).

Valve leaflets (33), sealing membrane (351, 354) and sealing loop (37) can not only prepared by biomaterials but synthetic macromolecular materials. For example, valve leaflet (33), sealing membrane (351, 354) and sealing loop (37) prepared by biomaterials are weaved in the stent (10); while self-expanding valve stent (1) can form a complex without suture, which is prepared by the synthetic polymers. Therefore, it can reinforce the intension of valve stent (1) and be no sharp dead side between the valve leaflet (33) and sealing membrane (351, 354).

Radial deformable self-expanding network stent (10) with centre-hollowed tubule-shaped network, is made up of elastic materials. Without external force, stent expands and is in the expanding state. Under external force, the stent is compressed radially and is state of compression. No matter in nature or expansion, self-expanding network stent (10) can be divided into three segments: the upper segment (13), the middle segment (15) and the upper segment (18).

As to the aortic valve, in case of the reverse blood flow approach adopted by the present invention, the upper segment (13) is the near end for operator, while in case of same blood flow approach, it is the distal end. The upper segment (13), cooperated with ascending aorta, comprises annular outline wrapped on long axis xx. In the nature or expansion, the upper segment (13) can be tubule-shaped and funnel-shaped, respectively. As it is funnel-shaped, the small-diameter end is near to the joint area (133) of the upper segment (13) and the middle segment (15), while the large one near to the lower segment (134). The length of lower segment (13) is change by the need. The deformable units (101) in the tips of the lower end (134) can be in either a same surface or a different one, and the tips of lower ends (134) in lower segment (13) can connect with the deformable unit by arched line-inflextions (102) or separate from the unit (101) by sealed line eye (103).

The middle segment (15) located in the middle segment of self-expanding network stent (10) is suitable to coronary cusp of the aortic root and aortic valve leaflet, which is changed from 15 to 30 mm for the demand. In the nature or expansion, the intermediate segment 15 can be divided into three types: 1. if annular outline structure wrapped axis is based on axis xx as the long axis: the structure contains tubule-shaped structure (151) and drum-shaped structure (152). 2. The middle segment 15 bears radial protrusion structure (153), if annular outline structure wrapped axis is based on long axis xx and compound structure with radial protrusion outline is based on side axis ax, bx and cx. 3. Inner and outer two-layer structure: the above outline frame comprises tubule-shaped (151), drum-shaped (152) and inner layer of stent body (154) with compound structure of radial protrusion framework. Outer frame out of the stent body 154 include the outer annular framework (155) and the outer tongue framework (156). Internal layer of stent body (154) connect with in the lower segment (13) or the joint area (133) of the lower segment (13) and the middle segment (15). In middle segment (15), certain deformable unit (101) with sealed line eye (103) can be separated from the other deformable units (101).

There are 6 types of stent about artificial heart valve stent (1) in this invention as follows:

Referring to FIG. 1 and combining with FIG. 1a, stent in FIG. 1 is the first type of stent, wherein middle segment (15) of the stent is the tubular shaped outline (151) wrapped by the long axis xx, while the middle segment of tubular-shaped (151) has a stent opening (158).

Reference to FIG. 2, the second type of stent is shown in FIG. 2, wherein the middle segment (15) of the stent is the drum-shaped outline (152) wrapped along the long axis xx. The largest diameter in of the drum-shaped outline (152) is in the middle segment, which is larger than the outer diameter of joint area (133) and (183), while the middle segment of drum-shaped (152) has a stent opening (158).

Reference to FIG. 3, from FIG. 3a to FIG. 3f, FIG. 3 is the third type of stent, wherein the middle segment 15 is a compound structure, including of based on the tubule-shaped outline (151) wrapped along the long axis xx, or the slight drum-shaped outline (152) wrapped along axis, one or more radial protrusion structure (153) based on the side axis ax, bx and cx in the outer surface, which extend outside. The side axis ax, bx and cx are vertical to the long axis xx and distributed by 120° rotary-angle. The radial protrusion structure (153) distributed by 120° rotary-angle is applied in the cooperation with coronary cusp or aortic valve leaflet. The radial protrusion structure (153), a part of the stent, with a larger outer diameter than (157), has a large stent opening (158) in the center. And the periphery (159i, 159o) connect with the rotary outline stent wrapped along axis. The middle segment 157x of outer radial protrusion framework in the periphery (159i, 159o) has small outer diameter. The periphery (159i, 159o) are divided into the upper periphery (159i) and the lower periphery (159o) by the commissure (160) as the boundary. The lunate periphery (159i) form a combined line of valve leaflet (331), which connects with valve leaflet (33). Two nearby radial protrusion structure (153) connected at the commissure (160), which overlap into one commissure. The middle segment (157x) of commissure (160), with small diameter, forms valve leaflet commissure (332). Aortic valve distributes 1-3 leaflets by 120° rotary-angle, while there is at least one leaflet in frame (153). In FIG. 3, it is shown that there are stents with three frames (153).

Reference to FIG. 4, combining with FIG. 4a, FIG. 4 is the fourth type of stent, wherein the middle segment (15), with tubule-shaped dual layer structure, contains inner-layer stent (154) and outer annular framework structure (155). Inner-layer stent (154) connects with outer annular (155) in the lower segment (13) or the joint area (133), which is named as stationary edge (161). The outer annular structure (155) exhibit free or active, terminated in the joint area (183), which is named as free edge (162). In nature or expansion, the inner layer of stent parallels to outerlayer of annular structure (155). While inner-layer stent (154), in compression, based on axis of stationary edge 161, can near to outer annular framework structure (155) via radial compression, or can extend far from inner-layer stent (154) to show as the funnel-shaped opening (184) by removal of centripetal force.

Reference to FIG. 5, combining with FIG. 5a and FIG. 5b, FIG. 5 is the fifth type of stent, wherein the middle segment (15) has inner and outer dual-layer compound framework. The inner-layer stent (154) of the rotary outline wrapped along axis (151) or (152) has a free tongue 156 wrapped by the single net line based on the side axis of dx, ex and fx in the outer surface, which is from the joint area (133) to the joint area (183). Moreover, the side axis of dx, ex and fx distributed by 120° rotary-angle, are vertical to the long axis xx. Three free tongue (156) distributed by 120° rotary-angle can be used in the cooperation of coronary cusp or aortic valve leaflet. Free tongue (156) is a part of stent, and a part of periphery in free tongue (156) like the lower periphery connects with inner-layer stent body (154), named as stationary edge (163). However, another part exhibited free or active is named as free edge (164). The two stationary edges (163) nearby the free tongue 156 encounter at the joint point (165), and the joint point (165) and commissure (332) is in the same rotary surface. While the inner-layer stent body in compression is based on axis of stationary edge (163), free tongue (156) can be radially compressed to inner-layer stent body (154), or can extend far from inner-layer stent body (154) to the funnel-shaped opening (184) by removal of centripetal force.

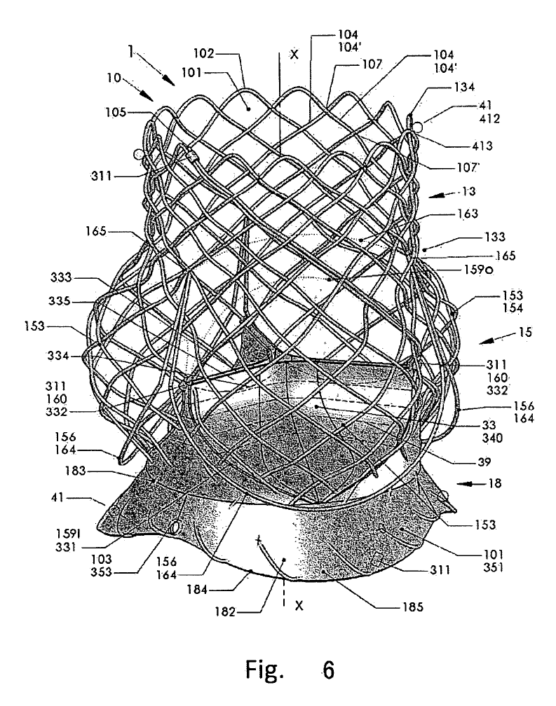

Reference to FIG. 6, FIG. 6 is the sixth type of stent, wherein the middle segment (15) is radial protrusion framework (153) in FIG. 6 and outer free tongue (156) in FIG. 5. Radial protrusion framework (153) and outer free tongue structure (156) exist in the place of same angle. The free edge (164) overlaps with periphery of (159i) and (159o) of radial protrusion framework (153), at least with the lunate upper periphery (159i) on the two parallel curved surfaces.

Further reference to FIG. 1-FIG. 6, the upper segment (18) cooperates with loop of aortic valve. As to the aortic valve, the upper segment (18), in case of the reverse blood approach, is the near end of stent for operator, while in case of same blood approach, it is the distal end. The upper segment (18) with the rotary outline wrapped along long axis xx can be tubular net-shape (181) (reference to FIG. 1 and FIG. 5) and funnel-shape (182) (reference to FIG. 2, FIG. 3, FIG. 4 and FIG. 5) in nature or expansion. Tubular net-shape (181) 181 is the extension from the tubule-shaped part of middle segment (15) to upper tip (184), while funnel-shape (182) is the extension from the funnel-shaped part of middle segment (15) to upper tip (184). The small opening of funnel-shape (182) is near to middle segment 15, while the big one is upper tip (184). The diameter of upper tip (184) in the upper segment (18) is far larger than diameter of (183), which is the joint area of upper segment (18) and middle segment (15). The length of upper segment (18) is normally less than 20 mm for the demand, so it can not disturb the mitral valve. No matter what is the project of either tubule-shaped (181) or funnel-shaped (182), upper segment (18) and the deformable unit (101), in the upper tip (184) of upper segment (18), are in the same surface. For example, the upper tip (184) of upper segment (18), existing with three hemispherical radial protrusion framework (153) synchronously, are in the different surface. Because of the shorter segment (18) corresponding to radial protrusion commissure (160) or valve leaflet commissure (332) and the longer segment (18) related to (157x) in the middle segment (153), the upper tip (184) of upper segment (18) is corresponding to trefoil wavy opening (185) of radial protrusion framework (153). In the upper tips (184) of the upper segment (18), the deformable unit (101) can be connected by arched line-inflextions, and can be separated from the deformable unit (101) with sealed line eye (103).

This invention adopts the self-expanding network stent (10), the above outline of which is in nature or expansion. The self-expanding network stent (10) is made up of elastic materials. And the known elastic biocompatible materials include Nitinol, Phynox, L605, et. al. It is difficult to prepare balloon expanding stent by elastic materials, because the above outline of stent demands special figure by expansion. Moreover, the preparation of self-expanding network stent (10) can not only be weaved by elastic lines, but also can be prepared by incising elastic pip.

The weaving method of the self-expanding network stent (10) adopts the basic methods as follows:

Reference to FIG. 1a, FIG. 4a, FIG. 5a, FIG. 1 and FIG. 6, before the weaving, at first the method is to prepare the internal mole fitted with the expanding stent, and then weaved by elastic braided line (104) along outline of internal mole. Weaving line first from one of tip points (105) and (156) in weaving line (104), extends helically along the above outline (151), (152), (153), (154), (155), (156), (181) or (182), while it approaches to tips of stent (134) and (184), then extends helically by the reverse direction along the same specific outlines. All deformable units (101) are prepared by repeating the steps, and it will ends at one tip point of (105) and (106) like 105, or outer of (105). The same single line (104) connects with two line-segments (104′) at the up and down cross point (107), where there are four nearest cross points (107′) with reverse location. A deformable unit (101) comprises a quadrilateral or rhombus, which is made up of four lines (104′) and four cross points (107), (107′). Deformable Unit (101) with four sides or stent weaved by deformable units (101) with four sides compress radially with transformation, transformating by extension along axis. Single weaving line (104) approaches to tip of stent, like the upper end (184) and lower end (134) or deformable unit (101), then turns reversely to form an arched line-inflextions (102), less than 360°. Sealed line eye (103) is formed by weaving line (104) of arched line-inflextions (102) re-rotated 360°. Sealed line eye (103) can be in or between in the ends of stent like the upper end (184) and lower end (134). One or more sealed line eye (103) can exist in each segment of line. Sealed line eye (103) can be in the same outer profile or section of stent, and in the outer or inner of the vertical surface (radial surface), even between the two ones. However, in the stent tips, for example like arched line-inflextions (102) of the upper end (184) and lower end. (134), sealed line eye (103) can be in the same surface or not. As to valve stent of tricuspid, it is available as the number of deformable unit along the girth is multiple of three, but the number of deformable units along long axis, divided by number of units along girth, may be a fraction, not be an integer. When the terminal point (106) of single weaving line reaches to beginning point (105), the weaving can be repeated after weaved a complete stent, including of: 1. complete repeat in the all area, it can form the stronger radial intensity of stent, in the dual line-segments or above; 2 repeat in the local of stent, such as upper segment, middle segment or lower segment, it leads to the enhanced local radial force in the dual line-segments or above. Furthermore, line-segments from dual to multiple approach or overlap to form different units (101), including of the big opening (158). Stent weaved by single line can also be weaved by multiline, which can be weaved by two or more same or different lines. Although each single line can form only one stent, two or more stents can overlap to form a compound stent. Single line can be different thickness and materials, for example, one of them can be made up of x-ray opaque markers, such as gold, tungsten, platinum, tantalum, et. al.

The Weaving Method of First Type:

The first method of the man-made heart valve stent (1):

The weaving method is same to the basic method, which is about axis-wrapped rotary outline stent of tubule-shaped (151) and (181) along long axis xx.

The Weaving Method of Second Type:

The knitting method of axis-wrapped rotary outline stent of the lower segment (13) with tubular net-shape based on axis xx, the middle segment (15) with drum-shaped (152) and the upper segment (18) with funnel-shape is same to the basic method, and the length of each segment of weaving line (104) is same from upper end (184) to lower end (134).

The Weaving Method of Third Type:

Based on the above two methods, tubular net-shape (151) along long axis xx, axis-wrapped rotary outline (152), one or more radial protrusion framework (153) along the side axis of ax, bx and cx in the outer surface of middle segment (15) extend outside to form a compound stent, which is similarly weaved by the basic method. Stent, with radial protrusion framework (153) in the middle segment (15), is weaved by single weaving line (104), which is originated through the different area of three half-ball radial protrusion structure (153) from lower end (134), such as middle segment (157x) or commissure (160), and end at joint area (183) of upper segment (18) and middle segment (15). However, length of each segment from the above steps is different and nearby deformable units are not in the same size. Noticeably, the slippage, between cross points (107) and (107′), ensures the radial compression and expansion of stent and radial protrusion structure (153). Because of existing with three radial protrusion structure (153), upper segment (18) of funnel-shape (182) relating to commissure (160) and (332) are shorter, while upper segment (18) of funnel-shape (182) corresponding to middle segment (157x) are longer. As a result, upper segment (18) of funnel-shape (182) is three trifoliate wavy opening (185) corresponding to three radial protrusion structure (153). Furthermore, weaving line, in the longer segment 18, passes through the small outer diameter of commissure (160) or (332), while it passes through the big one of middle segment (157x) of radial protrusion structure (153). Therefore, each length between protrudent structure (153) and tip (134) are same no matter that it is in expansion, compression or not. In expansion, opening (184) shows that three 185 are consistent with (153). In compression and extension on axis, segments of line slip nearby the commissure, (153) and (185) disappear, while deformable units of (184) are parallel. Single line (104) can not only knit a single-layer stent (10) but also a multilayer tridimensional stent.

The weaving method of fourth type:

Single line (104) knitting a single-layer network stent (10), locally repeat in situ with another segment (104′) in the same weaving line (104). Extended to middle segment (15), single line (104) separates from inner-layer stent (154) and singlely knits outer annular framework (155), then return to repeat in situ in the lower segment (13). Based on the above steps, line 104 repeat to knit between lower stent (13) and outer annular structure (155) in the middle segment and make a 360° rotary angle, finally forms outer annular structure (155) as shown in FIG. 4a, with two inner and outer dual-layer stent (154) and (155) in the middle segment. The joint of the dual layer is stationery edge (161), which is from the outer annular structure (155) to the joint area (183). The outer annular structure (155) is benefit for implantation. The inner-layer stent (154), in compression, is based on axis of stationary edge (161), while outer annular structure (155), radially being compressed to inner-layer stent (154), or apart from inner-layer stent (154) without centripetal force, is shown funnel-shaped. Independent from the compression and expansion of inner-layer stent (154), the annular structures (155) can have an effect of fixation and location. Furthermore, in expansion, inner-layer stent (154) and outer annular structure (155) can be stuck to the outer surface of inner-layer stent (154), and also extended to outer surface as shown in funnel shape. The proportion of unit CN of girth and units of axis is fraction, which are of outer annular structure (155) about repeat number of dual segment line in lower segment (13). Remarkably, outer annular structure (155) can not only be weaved by the same line (104) of inner-layer stent (154), but also by the different lines.

The weaving method fifth type:

After Single line (104) knitting a single-layer network stent (10), in the lower segment 13, another segment (104′) of the same line repeats in-situ and the stent turn a 600, then single line (104′) in the middle segment (15), extends and separates from stent (154), finally returns to repeat in situ of lower segment (13) by a half of arched line (166) or an complete arched line (166′). Therefore, the angle between entry (167) and inlet (167′), in the single line (104), can be 120°. Based on the above steps, the outer free tongues (156) repeat for 3 times to form the structure of (156) as shown in the FIG. 5a. However, the middle segment in the inner-layer stent (13) and outer free tongue (156) form dual-layer stent framework. Juncture of the two layers is a stationery edge (163), which is from joint area (133) to joint area (183), where the two stationery edges (163) of outer free tongue (156) have a same commissure (163). The outer free tongue (156) under radial compression can help the implantation. In stent's compression, outer free tongue (156), based on the axis of commissure (163), can approach the stent by the compression without inner-layer stent (154), or exhibit a funnel-shape and far from stent by the release of radial expansion without the centripetal force. Before the expanding stent (154), outer free tongue (156) can reach the natural valvar bag of aortic valve for a fixed use. No matter stent in compression or expansion, this outer free tongue (156) can be either radially expanded or compressed. These outer free tongues (156) enter the natural valvar bag and press on the bottom of valvar bag and natural valve commissure. In the time of closed valvar leaflet in diastole, blood flow reversely, while the outer free tongue (156) has a use for fixation and prevent valve stent from flowing into left ventricle by the blood. In compression of inner-layer stent (154) and outer free tongue (156), outer free tongue (156) can not only be stuck on the outer surface of stent but also extended to outer surface of stent. The proportion of unit CN of girth and units of axis is integer, which are of outer annular structure (155) about repeat number of dual segment line in lower segment (13), leading to the return of single line in the original points (105), (106). There are a half-arc (166) or annular (166′) with an arc more than 360° between weaving out point (167) and in point (167′), which can not be completely free but also be reweaved into in the lower segment of stent. The outer tongues (156) can be a part of the entire stent and can be distributed by two or three with rotary angle 120°. While the outer tongues (156) normally are a lunate arc, where the two tips of arched line connected with stent. Furthermore, there is other project deformed by outer tongues (156), as follows: 1. form a small loop to enhance deformed elastic force via weaving a 360° curve in the arched roof, 2. form a big loop in the arched roof, with a nearly same diameter of half arc, 3. weaving into the stent by the segment of big loop. However, the elastic force of outer tongues (156) is less than stent (154), because of lesser lines, while the small elastic force in intravascular cavity of outer tongues (156) can not hinder the stent expansion. Moreover, the size and figure of section, in outer tongues (156) and stent are same in expansion. Remarkably, the outer tongues (156) can not only be weaved by the same line 104 of inner-layer stent (154), but also by the different weaving lines.

The weaving method of the sixth type:

To weaving the radial protrusion structures (153) mentioned in the third method and the outer tongues (156) demonstrated in fifth method, stent can contains radial protrusion structures (153) and the outer tongues (156) with same size, figure, location and amount. After radial compression, the outer tongues (156) first release expansion, then intervene into the natural valve cup corresponding to the suitable the natural valve cup, in order to fix a rotary position and axis position, then the radial protrusion structures (153) and stent expand. Furthermore, the elastic force of outer tongues (156) is less than stent (154), because of the fewer lines, while the small elastic force in intravascular cavity of outer tongues (156) can not hinder the stent expansion. Radial protrusion structures (153) and the outer tongues (156) exhibit a use of fixation, while a sealing use by clamping natural valve medially.

In this invention, arched line-inflextions (102) and sealed line eye (103) can be formed by cutting tubule-shaped materials, while annular structure (155) and the outer tongues (156) can be prepared by the same materials and methods, respectively. Then the latter two structures can be jointed together.

Further reference to FIG. 1 to FIG. 6, an artificial heart valve (1) in present invention is designed with x-ray opaque markers, including of dot-shaped marker (311) and line-shaped marker (312).

Dot-shaped x-ray opaque markers (311), availably existing in tubule-shaped, put on the one or more weaving line (104) with same axis, is one or more x-ray opaque markers in the lower segment (134) of stent, while there are one or more x-ray opaque markers in the upper end (184) of stent or the joint area (183) of upper end and middle segment, near to the bottom of valve-leaflets cup. Even there is one or more x-ray opaque markers (311) in the middle segment (15) of stent, whose position can be located in the commissure (160), which is in the juncture of two radial protrusion structures (153), to be similarly equal to the location of two nearby commissures (332).

Reference to FIG. 5, originated from combined line (183) and ended in middle part of intermediate segment (157), a line of x-ray opaque markers can be prepared two wavy-shaped or three which is connected head and tail. However, the line of x-ray opaque markers can shuttle in the weaving net line (104) of stent, near to combined line of valve leaflets (331) of stent. Finally, triwavy marked line in the stent can be fixed on the biovalve leaflets.

Different x-ray opaque markers-x line can be prepared by the biocompatible heavy metal of gold, tungsten, platinum, tantalum, et. al.

Further referring from FIG. 1 to FIG. 6, valve leaflets (33), with two or three valve leaflet in an artificial heart valve stent (1) of the present invention, can be distributed by 120° rotary angle. Each valve leaflet cup comprises free side (333) and closed side (334), leading to a closed area (335) between free side (333) and closed side (334). Valve leaflets existing arched, can be divided into ascending and descending area, while the bottom of cup low to combined line of stent (331) form in the joint of stent and valve leaflet. Commissure (332), formed by the two nearby connected line, is in cross points (107, 107′) of weaving line (104). Moreover, commissure (332) can be corresponding to the same level of closed side (334). Valve leaflets, made up of soft materials, are close in nature. As a result of the linkage about free side (333) and closed side (334), the close valve can not pass the blood through. In diastole time, valve is closed of tighter by internal vasodilator press. However, in systole, blood, passed through valve leaflets (33) made up of biomaterials or synthetic materials, leads valve leaflets (33) can be stuck in the stent or inner vascular wall. The latter one can be elastomer, such as silica gel or polyurethane. One reinforced fibre (39) or more, in the synthetic valve, is originated from the two different valve leaflets of commissure (332) or combined line (331), and is ended in the stent (10). Reinforced fibre (39), chiefly in the side of aortic section 340, makes the surface of valve leaflet to be wirelike, while the side of ventricle in valve leaflet is mill finish.

Further reference to FIG. 1 to FIG. 6, sealing membrane can be designed in the valve stent (1), including of sealing membrane (351) and intermediate segment of sealing membrane (354).

Sealing membrane (351), wrapped in the tubule-shaped (181) or funnel-shaped opening (182), extend along the upper direction of stent to form soft membrane (352) without support of stent. However, it can also extend to the combined line of valve leaflets (331). In upper tip (184), arched line-inflexion (102) and sealed line eyes (103), there is at least one sealing membrane eye (353), connected with both inside and outside, to pass through stay guy (70) of implantation device (2). As a result, the upper sealing membrane (351) can ensure the leakage of blood through valve stent (1) in systole, while edge of soft membrane (352) can ensure the contact with natural mitral valve leaflet without injury.

Top sealing membrane (351) extends along upper from combined line of valve leaflets (331) to form middle segment of sealing membrane (354), which is wavy membrane with equal width. However, there is no membrane in the middle segment (157x) of radial protrusion structures (153). Wavy membrane, narrow in the commissure (160, 332), ensure the blood flow to coronary vein. In diastole, middle segment of sealing membrane (354), approaching to vascular wall for the impact of returning flow, ensure blood flowing to left ventricle without leakage from an artificial heart valve stent (1). Moreover, there is no sealing membrane (354) from the edge of (354) to the upper segment of stent, leading to ensuring the blood flow to side as perfusion of coronary vein and intervention of coronary vein in diastole.

The lower segment (13) without sealing membrane ensures blood perfusion of coronary artery bypass opening.

The cross points (107, 107′), without sealing membrane in the line of deformable unit (101), comprise elastic synthetic materials.

Sealing membrane (351, 354) can be biomembrane or synthetic membrane, while the former one can exist in the inboard, outboard or both two.

351,354 can be elastomer such as silicone gel, bundling the stent in the center.

Further referring from FIG. 1 to FIG. 6, sealing membrane (351, 354) with reinforced fibre (39), show annular placement and connect with stent. Reinforced fibre (39) can in the edge of synthetic sealing membrane, such as edge of soft membrane (352) and the intermediate segment of sealing membrane (354). Synthetic sealing membrane can be made up of macromolecular materials, such as silicon gel, latex and polyurethane. In radial compression, deformable unit, surrounded by elastomer, can extend along axis xx, or shrink along transverse axis. Extension along axis xx makes elastomer longer, and will be primary length by removal of outside force. After compression, stent extend, while materials flow to two sides, leading to the decrease of each section, which is benefit for reducing the outer diameter of valve stent in compression.

Reference to FIG. 3, in this invention, an artificial heart valve stent (1) is designed with sealing loop (37), which is a soft tubular net-shape loop, surrounded by the stent a circle, located in outside stent of the joint area 183 between upper segment 18 and middle segment 15 in the stent, and shown in triwavy shape along combined line (331) or circular shape along axis xx. Tubule-shaped structures can be sealed or half-opened. However, half-opened sealing loop (37) comprises a dot-shaped opening (373) (reference to FIG. 3f), opposite to inner or outer surface of valve stent (1), while slot-shaped opening (373′) (reference to FIG. 3f) opposite to inner surface. The tubule-shaped loop, prepared from biomaterials or synthetic materials, connects with sealing membrane (35). Although stent stick to vascular wall after expansion, sealing loop (37) can be compressed to fit with stent and fill up the gap between stent and vascular wall.

In this invention, an artificial heart valve stent (1) adopts the elastic synthetic membranes with reinforced fibre (39) intramembrane, which are prepared by elastic materials, with reinforced fibre (39). Compared with biovalve leaflets and sealing loops prepared by biomaterials, synthetic valve leaflet (33) and sealing membrane (351,354) can be designed with reinforced fibre (39). Synthetic valve leaflets, with one or more reinforced fibre (39), are originated from commissure (332) or combined line (331), connected in stent (10), while reinforced fibre (39) can be in free edge (333), chiefly in the lower section (340), which is wirelike drawn grain in the lower section (340) of aorta and mill finish of (341). Materials of reinforced fibre (39) consist of terylene fibre, polyethylene fibre with high molecular weight, nylon, carbon fiber et al. which cannot selectively enhance the intension of synthetic membrane but also the intension between membrane and stent. Moreover, reinforced fibre (39) can be on the x-ray opaque markers (311, 312).

Further referring from FIG. 1 to FIG. 6, an artificial heart valve stent (1) in the present invention is designed with flexible connecting loops (41). In the arched line-inflextions (102) or sealed line eye (103), or in the cross points (107, 107′), originated and ended by the different commissure (332) or combined line (331) in the same valve leaflet, flexible connecting loops (41) can be weaved by soft lines, which are prepared by terylene, polyester, polypropylene glycol, et. al. Soft lines first form a loop (412), with different size of loop and length of line. However, two tips, in another side of (412), tie a knot (413) and connect with it immobilly. Stayguy (70) in implantation device can pass and slip through flexible connecting loops (41), and can compress stent, because flexible connecting loops (41) limit hunting range of stayguy (70) and prevent the dislocation.

In a word, the artificial heart valve stent of this invention has traits and advantages, as follows:

1. Designed with Radial Protrusion Structures (153)

In middle segment (15) of valve stent, the ball-section of lower segment (13) and (18) can be divided into one or more radial protrusion structures (153), which is shape of upper spherical surface, or parabolic curved surface et al. In the stent, radial protrusion structures (153) of the valve stent (1) is a part of stent (10), which are made up of the same single line (104) and perfectly distributed into three half-sphericity radial protrusion structures (153) by 120°. Moreover, because diameter of the middle part (157x) in the middle segment of three radial protrusion structures (153) is larger, which can be benefit for fixation and location along or around axis xx. Compared with the valve stent (1) in cylinder-shape (151), radial protrusion structures (153) stick to the vascular wall, while on the same surface, two nearby radial protrusion structures (153) connect in the commissure (160) to form a valve leaflet commissure (332). Two nearby 153 are adducent in the commissures (160) and (332), with a small diameter compared with diameter of the middle part (157x) in the middle segment. As result, in working state, stent with a big diameter has a small valve leaflet but has enough opening surface may reduce the tension of valve leaflet; decrease the injuries of valve leaflet (33) in valve leaflets commissure (332), even more switching on blood flow without contact of stent (10), valve leaflet (33) will have no abrasion caused by the collision with stent; in the same thickness, the diameter of valve leaflet (33) decreases, which is benefit for radial compression. Lunate upper periphery (159i) form combined line of valve leaflet (331) connected with valve leaflet (33). Deformable units of radial protrusion structures (153) has the different lengths in the same surface, but the slippage, in the nearby weaving lines (104) of cross points of weaved stent (107), ensure the radial compression and expansion of stent and radial protrusion structure. However, the upper tip (184) located different surface, in the funnel-shaped (182) of upper segment, is three wavy edge (185) corresponding to three radial protrusion structures (153). Each segment of weaving line (104) has a same length from upper tip (184) to lower tip (134) in the stent. Furthermore, in radial compression and axial extension, nearby segment-lines in cross points slip, three radial protrusion structures (153) and three wavy edge (185) disappear, while deformable units in the upper segment are parallel, leading to cooperating stayguy in device (2) with arched line-inflextion (102) and sealed line eyes (103).

2. Designed with Outer Annular Structure (155)

Without sealing membrane, outer annular structure (155), passing blood through, cooperates with the special stayguy in implantation device and release earlier than stent (154), even more outer annular structure (155) has the effect of fixation and location.

3. Designed with Outer Free Tongue (156)

Without sealing membrane, outer free tongue (156), passing blood through, cooperates with the special stayguy in implantation device and release earlier than stent (156) has the effect of fixation and location. Noticeablely, the rotary relation between commissure (165) and commissure of valve leaflet (332) can be affirmed, such as in a same rotary surface.

4. Stent (10) Weaved by Single Elastic Line (104)

No matter what shape is, stent (10) can be weaved by single weaving line (104) with high integrity, burliness in mechanics and no jointing between among lines. The beginning point (105) and the ending point (106) of single line can connect with each other to joint and overlap, while the two tips (105) and (106) of weaving line in the single-line stent, between lower segment (13) and intermediate segment (15), can be opposite to the same direction, to direction of upper segment or lower segment. Single elastic weaving line (104) can wrap arched line-inflextions (102) and sealed line eyes (103), the latter ones can not be in the same outline curved surface or section, but also to be vertical to stent (radial section) opposite outer or inner, or between the two cases. As to valve stent leaflets of tricuspid, it is available for the number of deformable unit along the girth is multiple of three, but the number of units along long axis, divided by number of units along girth, may be a fraction, not to be a integer. Moreover, in the net stent (10), the same single weaving line (104) can form radial protrusion structures (153). Noticeably, the slippage, between cross points (107) and (107′), ensures the radial compression and expansion of stent and radial protrusion structures (153). Weaving the stent (10), the same single weaving line (104) can overlap at the same location one time or more, or repetitive weaving locally or completely in the stent, even more can weave outer annular structures (155) outer free tongues (156).

5. Designed with Sealing Loops (37)

Although stent stick to vascular wall after expansion, sealing loops (37) can be compressed to fit with stent and fill up the gap between stent and vascular wall.

6. Designed with Funnel-Opening in the Upper Tip of Valve Stent (1)

Upper tip (184), in the upper segment (18) is trifoliate wavy opening, corresponding to three radial protrusion structures (153). Sealing membrane (351) in the upper segment extends outside along the upper of stent to form soft membrane (352) without support of stent.

7. Designed with X-Ray Opaque Markers (311) and (312)

X-ray opaque markers (311) are located in commissure of upper tip, upper tip and valve leaflet. Annular tubulars of x-ray opaque markers, enchased on outside of single lines or overlapped multisegments, can be used for location of x-ray opaque markers and prevent dislocation of two lines or multiline in the same position and injuries of tissue from two line tips (105) and (106).

8. If Valve Stent (1) Prepared by Valve Leaflet (33), Sealing Membranes (351) and (354) and Sealing Loops (37), it Will Have Four Uses as Follows:

a. Valve leaflet (33) prevent from reflux, while sealing membranes (351) and (354) and sealing loops 37 and sealing loops (37) prevent leakage, as a basic use.

b. Valve stent (1) with good elastic deformation.

After cross weaving, weaving line (104) of self-expanding stent can form deformable quadrangular unit (101). The upper coat of cross point (107), or in quadrangle, is covered with synthetic sealing membranes (351) and (354). Stent and membrane, prepared by elastic materials, deform elastically together under the force of radial compression. Deformable units (101) extend along axis xx, while membrane in deformable quadrangular unit extends elastically along axis xx. In the balance with vascular wall or in working state, and before sealing membranes (351) and (354) and surface of elastic synthetic materials in the valve stent do not recover the original length and figure, rebound force caused by elastic deformation of elastic synthetic membrane, increases radial expanding force and rebound force along axis. After release of valve stent, valve leaflet and sealing membrane, prepared by elastic materials, can be super balloon-expansion, while stent can still be elastic deformation without injuries.

c. Elastic synthetic materials, packed on the metal stented line, prevent vascular epithelial unit growing on the metal line, leading to the separation valve from vascular wall for removal once more.

d. Compared with biovalve leaflets, synthetic valve leaflets and sealing membrane existing under 0° C. available, has no need of transport or particularly air parcel with special condition. For example, before equipment and compression, valve stent, prepared by Nitinol, can first be in the temperature under Af, while Nitinol turn from Austenitic to Martensitic. Materials turn soft and elasticity disappears, which is benefit for radial compression. However, after entering body, while temperature increase to 370° C., Nitinol returns to Austenitic and go back to be in super elasticity.

9. Designed with Reinforced Fibre (39)

Reinforced fibre (39), in the valve stent (1), selectively enhances intensity of sealing membranes (351) and (354) prepared by synthetic materials, and reduces the lancinated possibility of itself. Moreover, reinforced fibre (39), not only can reinforce valve leaflets (33) annularly to respect switch of valve leaflet, but can reinforce the free edge of valve leaflets (33) to prevent its lancinating; while reinforcement of commissure and combined line in the joint of synthetic valve leaflets (33), reinforced fibre (39) can solid the juncture and prevent lancinating; can reinforce between sealing membranes (351) and (354) and stent (10), even more, reinforced fibre (39) can fix the two line in cross 107.

10. Effect of arched line-inflextions (102) and sealed line eye (103) in valve stent (1) and cooperation with stayguy of stent with implantation device: Increasing the radial elastic force by arched line-inflextions (102) and sealed line eye (103) can reduce deformation of materials. Reinforced fibre, in the elastic synthetic membrane, can be fixed in arched line-inflextions (102) and sealed line eye (103). Moreover, sealed line eye (103) can fix the commissure (332). If sealed line eye (103) circumrotate along inner side, vertically to section, it will drive the commissure (332) an internal shift and will reduce the force of valve leaflet. Remarkably, inflextions (102) and sealed line eye (103), used in the cooperation with stented guy, can fix the valve stent (1) temporarily and can be compressed on the inner tubule (51). If guy pass through sealed line eye (103), it will not disconnect and move.

11. Designed with Flexible Connecting Loops

If stayguy of stent pass through flexible connecting loops (41) in valve stent (1), it will not disconnect and move.

### APPLICATION IN INDUSTRY

In this invention, compared with present technology, artificial heart valve stent adopting the above project has advantages and good effects as follows:

1. The shape, structure and function of artificial heart valve stent are optimized.

2. Stent, with deformable ability, can not cooperate with valve biomembrane but also synthetic one.

3. In the time of switching the valve, it can prevent friction with metal stent and blood leakage around valve.

4. After expanding release, it can be accordance with shape of vascular wall in axial and radial direction.

5. After implantation, it prevents slippage of valve caused by reverse blood as closing valve.

6. After expansion, valve will not generate paravalvular leak.

7. Radial protrusion structure in the valve can reduce the stress, borne by valve leaflet or joint between valve leaflet and stent.

8. Valve with radial protrusion structure can be exactly fixed and located along axial and rotary direction.

9. Valve with tongue structure can be exactly fixed and located along axial and rotary direction.

1. An artificial heart valve stent comprising:
a tubule-shaped stent being able to radial transformation between expanding state and compressing state;
the stent comprising an upstream segment, a middle segment and a downstream segment;
a plurality of transformable units among each netline of the stent;
a plurality of arc line crutches made up or formed at two ends of the stent and sealed line eyes which are separated from transformable units;
a valve leaflet which can switch for blood passing through of unidirectional provided on inside of a middle segment of the stent, a valve leaflet joint line being formed at the junction of the valve leaflet and stent, a crosslink of two adjacent said valve leaflet joint lines forming a valve leaflet joint point;
both inside and outside faces of the upstream segment of the stent being covered with sealing membrane that is extended to the middle segment of the stent; and
a plurality of X-ray opaque markers and flexible hitch loops provided on the stent.

2. The artificial heart valve stent according to claim 1, wherein the stent is made by interweaving of a same one of elastic metal line, two segments of the line can be slipped or rotated relating to each other at the crossing point thereof.

3. The artificial heart valve stent According to claim 1, wherein the middle segment of said stent can be deformed into at least one radial protrusion structure of outward protrusion on the basis of round tubular or slight drum-shape, one larger stent opening is provided in the centre of every radial protrusion structure, a half-mooned upward periphery and a half-mooned downward periphery are formed at the junction of said radial protrusion structure and stent itself, the half-mooned upward periphery is formed into the valve leaflet joint line connected with the valve leaflet, while said valve leaflet is corresponding to radial protrusion structure and is connected to half-mooned upstream periphery of the radial structure.

4. The artificial heart valve stent according to claim 3, wherein said radial protrusion structure of said stent middle segment is one.

5. The artificial heart valve stent according to claim 3, wherein said radial protrusion structure of said stent segment is two, said two radial protrusion structures are distributed by rotary angle from 90°-180°.

6. The artificial heart valve stent according to claim 3, wherein said radial protrusion structure of the stent middle segment are three, which are uniformly distributed along circumference of the net-shaped stent.

7. The artificial heart valve stent according to claim 3, wherein the upstream segment of said stent is horn-type.

8. The artificial heart valve stent according to claim 7, wherein the outer edge of horn-type upstream segment is provided with wave-shaped edge, corresponding to said radial protrusion structure of the middle segment.

9. The artificial heart valve stent according to claim 1, wherein the stent also comprises tubular net-shaped internal layer stent body, or the internal layer stent body with the radial protrusion structure, the stent body is connected with at least one outer layer tongue structure surrounded by net line, the outer layer tongue structure and internal layer stent body form a stationary edge at the downstream segment, or at the junction of the middle segment and downstream segment of said stent, and extend from stationary edge along the upstream segment to junction of the upstream and middle segments to form a free edge, moreover, the free edge overlaps with periphery of radial protrusion structure, at least with the half-mooned upstream periphery, on the two parallel carved surfaces.

10. The artificial heart valve stent according to claim 9, wherein the number of out layer tongue structure is three, which are uniformly distributed by circumference in a rotary angle way, along the interior layer stent body.

11. The artificial heart valve stent according to claim 9, wherein outer layer tongue structure is corresponding to interior layer radial protrusion structure at radial and axial directions, and they are provided on the same rotational angle.

12. (canceled)

13. (canceled)

14. (canceled)

15. (canceled)

16. The artificial heart valve stent according to claim 1, wherein said sealing loop is equipped at outside junction of upstream and middle segments of the stent, which is flexible half-open type tubule-shaped structure, on which a plurality of dot-shaped openings faced on outer face and inner face of membrane are provided, or a trough opening faced on inner face of the stent valve is provided.

17. A weaving method of the stent comprising preparing an internal mould fitted with the configuration in expanding state of the stent, an elastic metal line is used as weaving line, main weaving points are as follows:
A. weave spirally along exterior outlines of the internal mould with the weaving lines until all transformable units have been built to weave a complete stent body,
B. the different line segments of weaving line form an up and down cross points when meeting at an intersection, while the up and down locations of the same line segment, at their adjacent cross points are converse,
C. Deformable units surrounded by the different line segments of the weaving lines are quadrangle, weaving line turned at the two tips of the stent form arched line-inflextions or line bump,
D. according to the need, sealed line eyes are made by turning the weaving lines round at least 360° cyclically at two ends of the stent or other places,
E. for a stent with three radial protrusion structures, the number of deformable units located in a same radial plane of the stent are weaved into multiple of 3,
F. according to need, x-ray opaque markers are labeled on the weaving line of different places of the stent.

18. The weaving method of stent according to claim 17, wherein said sealed line eyes are weaved to be in same outline curve surface with the stent or are weaved to be vertical with the stent or to make any angle.

19. The weaving method of stent according to claim 17, wherein after has been weaved a stent body, the position of the local or all parts at the stent is re-weaved to form the stent of single layer multi-lines structure or two-layer structure or multilayer structure locally and completely.

20. The weaving method of stent according to claim 17, wherein the weaving lines are single elastic metal line.

21. The weaving method of stent according to claim 17, wherein the weaving lines are double lines or multiline, consisting of a plurality of elastic metal lines, comprising a single line made by x-ray opaque materials in them.

22. The weaving method of stent according to claim 17, wherein the weaving lines contains a plurality of single lines, each of which can be weaved into a stent, a plurality of stents are over lapped together to form a combined stent.

23. The weaving method of stent according to claim 17, wherein on the stent body weaved in step A, outer layer tongue structure can also be weaved, and the main points of the weaving of the lanque structure are as follows:
a. at the beginning weaving, lines are re-weaved from the downstream of the weaved stent body, as they are weaved to corresponding to the angle of 60° around the stent, the lines are separated from the stent body, and after the lines extended outward are weaved into a tongue structure, then enter the stent through turning around symmetric opposite direction to be re-weaved, when weaving is nearly one third of circum around the stent, let the weaving lines separate from the stent body, after the lines extended outward are weaved into a tongue structure, then they enter in the stent body through turning around symmetric opposite direction to be re-weaved, until they are weaved into three outer layer structures, finally a segment of the weaving line enters into the stent body weave repeatedly to the downstream port of the stent again,
b. controlling the out and in points of the weaving line extended from the stent body and entered in the stent body and making them locate in the same radial plane, and controlling the distance between the out-point and in-point, which is corresponding to one third of circum turning around the stent, and controlling the free edge of the tongue structure located in the junction of the upstream and middle segments of the stent body.

24. (canceled)

25. (canceled)

26. The weaving method of stent according to claim 23, wherein said main point a, when a tongue structure is wrapped by weaving lines, its arc-crown generate sealed line eyes around at least 360°, on the dual line segments of the line eye are put the x-ray paque maker loop.

27. The weaving method of stent according to claim 23, wherein said tongue structures and stent body are weaved by the same weaving line.

28. The weaving method of stent according to claim 23, wherein said tongue structures and stent body are weaved by different weaving line.
